(19) United States
(12) Patent Application Publication
(10) Pub. No.: US 2004/0158068 A1
(43) Pub. Date: Aug. 12, 2004

(54) Method for oxidising an aromatic aldehyde into the corresponding carboxylic acid

(76) Inventors: Roland Jacquot, Francheville (FR); Jean-Louis Grieneisen, Lyon (FR)

Correspondence Address: Jean Louis Seugnet, Intellectual Property Department, Rhodia Inc CN 7500, 259 Prospect Plains Road, Cranbury, NJ, 08512-7500, US

(21) Appl. No.: 10/477,298

(22) PCT Filed: May 7, 2002
(86) PCT No.: PCT/FR02/01566

(30) Foreign Application Priority Data
May 11, 2001 (FR) 01/06260

(51) Int. Cl. C07D 215/38; C07D 213/46; C07D 239/02; C07C 51/23

(52) U.S. Cl. 544/335; 544/406; 546/169; 546/315; 562/421

(57) ABSTRACT

The present invention concerns a process for oxidising an aromatic aldehyde to the corresponding carboxylic acid. The process of the invention for preparing an aromatic acid by oxidising an aromatic aldehyde consists of carrying out the oxidation of the aromatic aldehyde in a basic medium using molecular oxygen or a gas containing molecular oxygen in the presence of a catalyst, and is characterized in that oxidation is carried out in the presence of an effective quantity of a palladium and/or platinum based catalyst under conditions such that oxidation is carried out in a diffusion regime.

The present invention relates to a process for oxidising an aromatic aldehyde to the corresponding carboxylic acid.

More particularly, the invention relates to a process for oxidising vanillin to p-vanillic acid or 3-methoxy-4-hydroxybenzoic acid.

European patent EP-A-0 773 919 describes the preparation of vanillin or 3-methoxy-4-hydroxybenzaldehyde using a process consisting of reacting formol and guaiacol in the presence of sodium hydroxide to produce a mixture comprising o-hydroxymethylguaiacol (OMG), p-hydroxymethylguaiacol (PMG), 4,6-di(hydroxymethyl)guaiacol (DMG) then oxidising said mixture with oxygen in the presence of a palladium catalyst and a bismuth co-catalyst and then eliminating the carboxylic group located in the ortho position from the oxidation products containing it to obtain vanillin in a very good reaction yield.

At the end of the oxidation step, the following quantities of products are obtained:

| ortho series: | |
|---|---|
| RR o-vanillin (OVA) = | 1% |
| RR o-vanillic acid (AOV) = | 14% |
| para series: | |
| RR vanillin (PVA) = | 16% |
| RR p-vanillic acid (APV) = | 1% |
| di- series: | |
| RR o-carboxyvanillin (OCVA) = | 47% |
| RR 4,6-(dicarboxy)guaiacol (DCG) = | 10% |

In the process described in EP-A-0 773 919, selective oxidation to a carboxyl group occurs of the hydroxymethyl and formyl group located in the position ortho to a hydroxyl group. p-vanillic acid is only obtained in a very low yield of 1%.

Against all expectations, the Applicant has discovered that p-vanillic acid can be obtained by oxidising vanillin using the same type of catalytic system but under certain processing conditions.

It has also unexpectedly been discovered that the process of the invention can be generalised to the preparation of any aromatic acid from the corresponding aldehyde provided that the conditions defined in the invention are satisfied.

More precisely, the present invention provides a process for oxidising an aromatic aldehyde to the corresponding carboxylic acid, consisting of carrying out the oxidation, in a basic medium, of an aromatic aldehyde using molecular oxygen or a gas containing molecular oxygen in the presence of a catalyst, characterized in that oxidation is carried out in the presence of an effective quantity of a palladium and/or platinum based catalyst under conditions such that oxidation occurs in the diffusion regime.

In a preferred variation, the process of the invention further consists of adding, as an activator, metals from group 1b and 8 such as cadmium, bismuth, lead, silver, tin or germanium, preferably bismuth.

In particular, it has been discovered that a p-hydroxybenzoic acid can be obtained by oxidising a compound comprising a formyl group located in the position para to the hydroxyl group provided that the reaction conditions are controlled; they must be diffusion regime.

In the present text, the term “diffusion regime”, also known as “physical regime” denotes conditions that correspond to the conventional definition known to the skilled person.

Reference in this regard should be made to the work by J. RICHARDSON, Principles of Catalyst Development (1989), Plenum Press, New York, and by J. VILLERMAUX, Génie de la réaction chimique: conception et fonctionnement des réacteurs [Engineering the chemical reaction: reactor design and function] (1993), Lavoisier.

Diffusion regime conditions are such that the concentration of oxygen dissolved in the medium is close to zero.

In the following disclosure of the invention, the term “aromatic aldehyde” means an aromatic compound wherein one hydrogen atom directly bonded to the aromatic ring is replaced by a formyl group, and the term “aromatic compound” denotes the conventional notion of aromaticity as defined in the literature, in particular by Jerry MARCH, Advanced Organic Chemistry, 4^{th} edition, John Wiley & Sons, 1992, pp. 40 ff.

More particularly, the present invention is applicable to aromatic aldehydes carrying a free OH group or an OH group that is protected in the form of an ether.

Preferably, the substrate employed has general formula (I):

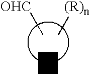

in which:

A denotes the residue of a cyclic group forming all or a portion of an aromatic monocyclic or polycyclic carbocyclic or heterocyclic system comprising at least one formyl group;

R represents a hydrogen atom or one or more substituents which may be identical or different;

n, the number of substituents in a cyclic group, is 5 or less.

The invention is particularly applicable to aromatic aldehydes satisfying formula (I) in which A is the residue of a cyclic compound, preferably containing at least 4 atoms in the cycle, preferably 5 or 6, optionally substituted, and representing at least one of the following cycles:

a monocyclic or polycyclic aromatic carbocycle;

a monocyclic or polycyclic aromatic heterocycle comprising at least one of heteroatoms O or N.

without limiting the scope of the invention, it can be stated that residue A, optionally substituted, can represent the residue:

1°—of a monocyclic or polycyclic aromatic carbocyclic compound.

The term “polycyclic carbocyclic compound” means:

a compound constituted by at least 2 aromatic carbocycles and together forming ortho- or ortho- and peri-condensed systems;

a compound constituted by at least 2 carbocycles only one of which is aromatic and together forming ortho- or ortho- and peri-condensed systems.

2°—of a monocyclic or polycyclic aromatic heterocyclic compound.

The term “polycyclic heterocyclic compound” means:

a compound constituted by at least 2 heterocycles containing at least one heteroatom in each cyclc at least one of the two cycles being aromatic and together forming ortho- or ortho- and peri-condensed systems;

a compound constituted by at least one carbocycle and at least one heterocycle at least one of the cycles being aromatic and together forming ortho- or ortho- and peri-condensed systems.

More particularly, optionally substituted residue A represents one of the following cycles:

an aromatic carbocycle:

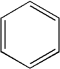

an aromatic bicycle comprising two aromatic carbocycles:

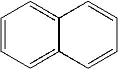

a partially aromatic bicycle comprising two carbocycles one of which is aromatic:

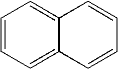

an aromatic heterocycle comprising 1 or more (2 to 4) heteroatoms:

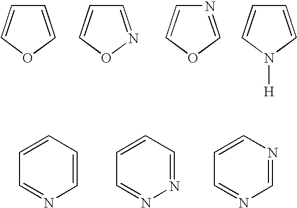

an aromatic bicycle comprising an aromatic carbocycle and an aromatic heterocycle:

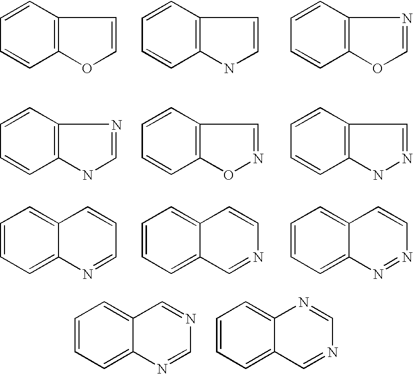

a partially aromatic bicycle comprising an aromatic carbocycle and a heterocycle:

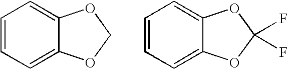

an aromatic bicycle comprising two aromatic heterocycles:

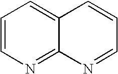

a partially aromatic bicycle comprising a carbocycle and an aromatic heterocycle:

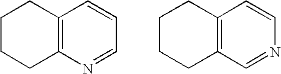

a tricycle comprising at least one carbocycle or an aromatic heterocycle:

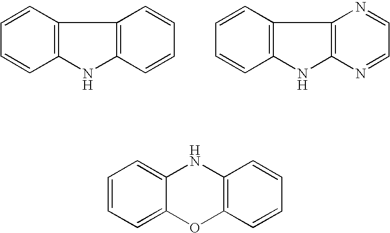

In the process of the invention, an aromatic aldehyde is used with formula (I) in which A represents the residue of a carbocyclic compound such as benzene or naphthalene or the residue of a nitrogen-containing heterocyclic compound, preferably pyridine, pyrimidine, pyrazine, quinoline or isoquinoline.

The aromatic compound with formula (I) can carry one or more substituents.

The number of substituents present on the cycle depends on the carbon condensation of the cycle and the presence or absence of unsaturated bonds on the cycle.

The maximum number of substituents that can be carried by a cycle can readily be determined by the skilled person.

In the present text, the term “several” generally means less than 5 substituents on an aromatic ring.

Advantageously, n is 1 or 2.

Examples of substituents are given below, but this list is not limiting in nature.

The present invention does not exclude the presence on the aromatic cycle of substituents of a different nature provided that they do not interfere with the reactions of the process of the invention.

Groups R, which may be identical or different, preferably represent an alkyl, alkoxy, alkenyl, alkenyloxy, hydroxyalkyl, alkoxyalkyl, cycloalkyl, cycloalkyloxy, aryl, aryloxy, arylalkyl, arylalkyloxy group, a hydroxyl group, a nitro group, a halogen atom, a halogeno group or a perhalogenoalkyl group.

Within the context of the invention, the term “alkyl” means a linear or branched hydrocarbon chain containing 1 to 15 carbon atoms, preferably 1 or 2 to 10 carbon atoms.

The term “alkenyl” means a linear or branched hydrocarbon group containing 2 to 15 carbon atoms, comprising one or more double bonds, preferably 1 or 2 double bonds.

The term “cycloalkyl” means a cyclic hydrocarbon group containing 3 to 8 carbon atoms, preferably a cyclopeniyl or cyclohexyl group.

The term “aryl” means an aromatic mono- or polycyclic group, preferably mono- or bicyclic containing 6 to 12 carbon atoms, preferably phenyl or naphthyl.

The term “arylalkyl” means a linear or branched hydrocarbon group carrying a monocyclic aromatic cyclic group and containing 7 to 12 carbon atoms, preferably benzyl.

Particularly suitable carbocyclic compounds for carrying out the process of the invention have formula (I) in which R, which may be identical or different, represents:

a hydrogen atom;

a linear or branched alkyl group containing 1 to 12 carbon atoms, preferably 1 to 4 carbon atoms, such as methyl, ethyl, propyl, isopropyl, butyl, isobutyl, sec-butyl or tert-butyl;

a linear or branched alkenyl group containing 2 to 6 carbon atoms, preferably 2 to 4 carbon atoms, such as vinyl or allyl;

a linear or branched alkoxy group containing 1 to 6 carbon atoms, preferably 1 to 4 carbon atoms, such as a methoxy, ethoxy, propoxy, isopropoxy, butoxy, isobutoxy, sec-butoxy or tert-butoxy radical;

a phenyl group;

a halogen atom, preferably a fluorine, chlorine or bromine atom;

a trifluoromethyl group.

In the heterocyclic aromatic compounds with formula (I), the preferred substituents are groups R, which may be identical or different, which represent an alkyl or alkoxy group containing 1 to 4 carbon atoms, a halogen atom, a halogeno group or a perhalogenoalkyl group.

More particularly, the process of the invention is applicable to aromatic aldehydes with formula (Ia):

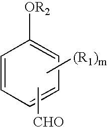

in which:

m is 4 or less, preferably 0 or 1;

R₁ represents a hydrogen atom or one or more substituents, which may be identical or different;

R₂ represents a hydrogen atom or an alkyl, alkenyl, cycloalkyl, aryl or arylalkyl group;

groups R₁ and R₂ and the 2 successive atoms on the benzene ring can together form a cycle containing 5 to 7 atoms, optionally comprising a further heteroatom;

two groups R₁ placed on two neighbouring carbon atoms can form a cycle containing 5 to 7 atoms together with the carbon atoms carrying them.

When m is 1 or more, two groups R₁ and the 2 successive atoms on the benzene ring can be bonded together by an alkylene, alkenylene or alkenylidene group containing 3 to 5 carbon atoms to form a saturated, unsaturated or aromatic cyclic group containing 5 to 7 carbon atoms, preferably a benzene ring.

Groups R₁ and R₂ can be bonded together and form an alkylene, alkenylidene or alkenylidene group containing 2 to 4 carbon atoms to form a saturated, unsaturated or aromatic heterocycle containing 5 to 7 atoms with-the two neighbouring carbon atoms that carry R₁ and OR₂. One or more carbon atoms can be replaced by a further heteroatom, preferably oxygen. Then groups OR₂ and R₁ can represent a methylenedioxy or ethylenedioxy group.

In formula (Ia), the cycle can optionally be substituted; examples of cyclic substituents that can be envisaged include substituents such as R₁, the meaning of which is given above for R in formula (I) for the aromatic aldehydes.

More particularly, the process of the invention is applicable to aromatic aldehydes with formula (Ia) in which R₂ represents a hydrogen atom or a linear or branched alkyl group containing 1 to 6 carbon atoms, preferably 1 to 4 carbon atoms, or a phenyl group.

In formula (Ia), R₂ preferably represents a methyl or ethyl group.

The aromatic aldehyde with formula (Ia) can carry one or more substituents R₁, more preferably one of the following atoms or groups:

a linear or branched alkyl group containing 1 to 6 carbon atoms, preferably 1 to 4 carbon atoms such as methyl, ethyl, propyl, isopropyl, butyl, isobutyl, sec-butyl or tert-butyl;

a linear or branched alkoxy group containing 1 to 6 carbon atoms, preferably 1 to 4 carbon atoms, such as methoxy, ethoxy, propoxy, isopropoxy, butoxy, isobutoxy, sec-butoxy, tert-butoxy;

a halogen atom, preferably a fluorine, chlorine or bromine atom, or

a trifluoromethyl group.

In formula (Ia), R₁ preferably represents a linear or branched alkoxy group containing 1 to 4 carbon atoms, preferably a methoxy or ethoxy group.

In formula (Ia), the formyl group is in the ortho, para or meta position, preferably in the meta or para position to a hydroxyl group, if present on the benzene ring.

Preferably, the present invention is applicable to compounds with formula (Ia) in which groups R₁ represent a hydrogen atom, a hydroxyl group or a linear or branched alkoxy group containing 1 to 4 carbon atoms; R₂ represents a hydrogen atom, a linear or branched alkoxy group containing 1 to 4 carbon atoms; groups OR₂ and R₁ can form a methylenedioxy or ethylenedioxy group; and m equals 0, 1 or 2.

Illustrative examples of compounds with formula (I) or (Ia) that can in particular be mentioned are: p-methoxybenzaldehyde, vanillin, o-vanillin, isovanillin, ethylvanillin, veratric aldehyde, piperonal, protocatechuic aldehyde and 2-formyl-6-hydroxynaphthalene.

Compounds for which the process of the invention are particularly advantageous are vanillin, ethylvanillin and veratric aldehyde.

The catalyst used in the process of the invention must be capable of operating in a diffusion regime.

To this end, the quantity of oxygen dissolved in the medium is limited by controlling different parameters of the process such as temperature, pressure and stirring rate. It is important that oxygen is consumed as soon as it arrives in the medium.

The catalyst used in the process of the invention is based on a metal M₁, which is palladium, platinum or mixtures thereof.

Preferably, platinum and/or palladium catalysts are used, in any available form such as: platinum black, palladium black, platinum oxide, palladium oxide or the noble metal itself deposited on different supports such as carbon black, graphite, activated charcoal, aluminas or activated silicas or equivalent materials. Catalytic masses based on carbon black are particularly suitable.

Generally, the metal is deposited in an amount of 0.5% to 95%, preferably 1% to 5% of the catalyst weight.

The quantity of catalyst used, expressed as the weight of metal M₁ with respect to that of the compound with formula (I) or (Ia), can vary from 0.001% to 10%, preferably 0.002% to 2%.

Further details regarding the catalysts can be obtained from U.S. Pat. No. 3,673,257, and French patents FR-A-2 305 420 and FR-A-2 350 323.

The activator can be selected from all those mentioned in the above patents. Preferably, bismuth, lead and cadmium are used as the free metal or as cations. In the latter case, the associated anion is not critical and all derivatives of these metals can be used. Preferably, bismuth metal or its derivatives is used.

A mineral or organic bismuth derivative can be used in which the bismuth atom has an oxidation number of more than zero, for example 2, 3, 4 or 5. The residue associated with the bismuth is not critical provided that is satisfies this condition. The activator can be soluble or insoluble in the reaction medium.

Illustrative examples of activators that can be used in the process of the present invention are: bismuth oxides; bismuth hydroxides; salts of mineral hydracids such as: bismuth chloride, bromide, iodide; salts of inorganic oxyacids such as: bismuth sulphite, sulphate, nitrite, nitrate, phosphite, phosphate, pyrophosphate, carbonate, perchlorate; and salts of oxyacids derived from transition metals such as: bismuth vanadate, niobatg, tantalate, chromate, molybdate, tungstate or permanganate.

Other suitable compounds are the salts of aliphatic or aromatic organic acids such as: bismuth acetate, propionate, benzoate, salicylate, oxalate, tartrate, lactate or citrate; and phenates such as: bismuth gallate or pyrogallate. These salts and phenates can also be bismuthyl salts.

Specific examples that can be cited are:

oxides: BiO; Bi₂O₃; Bi₂O₄; Bi₂O₅;

hydroxides: Bi(OH)₃;

salts of mineral hydracids: bismuth chloride BiCl₃; bismuth bromide BiBr₃; bismuth iodide BiI₃;

salts of inorganic oxyacids: basic bismuth sulphite Bi₂(SO₃)₃,Bi₂O₃,5H₂O; neutral bismuth sulphate Bi₂(SO₄)₃; bismuthyl sulphate (BiO)HSO₄; bismuthyl nitrite (BiO)NO₂,0.5H₂O; neutral bismuth nitrate Bi(NO₃)₃,5H₂O; double nitrate of bismuth and magnesium 2Bi(NO₃)₃,3Mg(NO₃)₂,24H₂O; bismuthyl nitrate (BiO)NO₃; bismuth phosphite Bi₂(PO₃H)₃,3H₂O; neutral bismuth phosphate BiPO₄; bismuth pyrophosphate Bi₄(P₂O₇)₃; bismuthyl carbonate (BiO)₂CO₃;0.5H₂O; neutral bismuth perchlorate Bi(ClO₄)₃,5H₂O; bismuthyl perchlorate (BiO)ClO₄;

salts of oxyacids derived from transition metals: bismuth vanadate BiVO₄; bismuth niobate BiNbO₄; bismuth tantalate BiTaO₄; neutral bismuth chromate Bi₂(CrO₄); bismuthyl dichromate ([BiO]₂Cr₂O₇; acid bismuthyl chromate H(BiO)CrO₄; double chromate of bismuthyl and potassium K(BiO)CrO₄; bismuth molybdate Bi₂(MoO₄)₃; bismuth tungstate Bi₂(WO₄)₃; double molybdate of bismuth and sodium NaBi(MoO₄)₂; basic bismuth permanganate Bi₂O₂(OH)MnO₄;

salts of aliphatic or aromatic organic acids: bismuth acetate Bi(C₂H₃O₂)₃; bismuthyl propionate (BiO)C₃H₅O₂; basic bismuth benzoate C₆H₅CO₂Bi(OH)₂; bismuthyl salicylate C₆H₄CO₂(BiO)(OH); bismuth oxalate (C₂O₄)₃Bi₂; bismuth tartrate Bi₂(C₄H₄O₆)₃,6H₂O; bismuth lactate (C₆H₉O₅)OBi,7H₂O; bismuth citrate C₆H₅O₇Bi;

phenates: basic bismuth gallate C₇H₇O₇Bi; basic bismuth pyrogallate C₆H₃(OH)₂(OBi)(OH).

Preferred bismuth derivatives for use in the process of the invention are: bismuth oxides; bismuth hydroxides; bismuth or bismuthyl salts of inorganic hydracids; bismuth or bismuthyl salts of inorganic oxyacids; bismuth or bismuthyl salts of aliphatic or aromatic organic acids; and bismuth or bismuthyl phenates.

A particularly suitable group of activators for carrying out the process of the invention is constituted by: bismuth oxides Bi₂O₃ and Bi₂O₄; bismuth hydroxide Bi(OH)₃; neutral bismuth sulphate Bi₂(SO₄)₃; bismuth chloride BiCl₃; bismuth bromide BiBr₃; bismuth iodide BiI₃; neutral bismuth nitrate Bi(NO₃)₃,5H₂O; bismuthyl nitrate BiO(NO₃); bismuthyl carbonate (BiO)₂CO₃,0.5H₂O; bismuth acetate Bi(C₂H₃O₂)₃; and bismuthyl salicylate C₆H₄CO₂(BiO)(OH).

The quantity of activator used, expressed as the quantity of metal contained in the activator with respect to the weight of metal M₁ used, can be varied between wide limits. As an example, this quantity can be as low as 0.1% and can attain the weight of metal M₁ used, or even exceed it without any problems. Advantageously, it is about 50%.

The pH is an important parameter in the process of the invention. It must be alkaline and is advantageously between 10 and 12.

Basic agents are employed, also alkaline or alkaline-earth bases; examples that can be cited are hydroxides such as sodium, potassium or lithium hydroxide.

Sodium or potassium hydroxide are used for reasons of economy.

The concentration of basic starting solution is not critical. The concentration of the alkali metal hydroxide solution employed is generally between 5% and 50% by weight.

The quantity of base introduced into the reaction medium takes into account the quantity necessary to form the salt of the carboxylic function that is formed and to form the salt of the hydroxyl function when the compound with formula (I) or (Ia) contains one.

If said compound has salt-forming functions other than a hydroxyl group, the quantity of base necessary to form the salts of all of the salt-forming functions is introduced.

Generally, the quantity of base expressed with respect to the compound with formula (I) or (Ia) is in the range 90% to 200% of the stoichiometric quantity.

The concentration by weight of compound with formula (I) in the liquid phase is normally in the range 1% and 40%, preferably in the range 2% to 30%.

In accordance with the invention, the oxidation temperature is preferably in the range 20° C. to 140° C., more preferably in the range 30° C. to 100° C.

Generally, atmospheric pressure is applied, but it is possible to operate at a pressure between 1 and 20 bars.

Regarding the stirring conditions, the skilled person is capable of determining them to maintain a diffusion regime.

By way of indication, it can be stated that in the case of a 3.2 litre reactor provided with a stirrer using 4 inclined paddles immersed in the reaction medium, the stirring conditions are advantageously in the range 500 to 700 rpm.

In practice one manner of carrying out the process consists of introducing the compound with formula (I), the basic agent, the palladium and/or platinum based catalyst, and optional activator in the proportions indicated above.

This implementation is entirely suitable when the compound with formula (I) carries a hydroxyl group.

When the compound with formula (I) or (Ia) carries a hydroxyl group in the protected form (ether), then in general, the-water, basic agent, palladium and/or platinum based catalyst, activator and compound with formula (I) or (Ia) are charged.

The reaction mixture is then heated in a stream of an inert gas (for example nitrogen) to the desired reaction temperature, then oxygen or an oxygen-containing gas is introduced.

The mixture is then stirred at the desired temperature until a quantity of oxygen corresponding to that necessary to transform the formyl group into a carboxyl group has been consumed.

At the end of the reaction, which preferably takes between 30 minutes and 6 hours, recovery takes place of the carboxylic compound with formula (III), formula corresponding to formula (I), preferably (Ia) in which the CHO group is replaced by COOM; M representing the cation corresponding to that of the base employed.

Then after cooling if necessary, the catalytic mass is separated from the reaction medium, for example by filtering.

In a subsequent step, the resulting medium is acidified by adding a protonic acid of mineral origin, preferably hydrochloric acid or sulphuric acid or an organic acid such as methanesulphonic acid to obtain a pH that is lower than the pKa of the acid obtained.

The concentration of acid is of no consequence; preferably, commercially available forms are used.

Acidification is generally carried out between ambient temperature (usually between 15° C. and 25° C.) and 100° C.

The precipitated aromatic acid is then recovered using conventional liquid/solid separation techniques, preferably by filtering.

It satisfies formula (IV), which corresponds to formula (I), preferably (Ia) in which the CHO group has been replaced by COOH.

The process of the invention is of particular application to the preparation of the following carboxylic acids: p-methoxybenzoic acid, p-vanillic acid, o-vanillic acid, isovanillic acid, 3-ethoxy-4-hydroxybenzoic acid, veratric acid, piperonic acid, protocatechuic acid and 2,6-hydroxynaphthalenecarboxylic acid.

The reaction can be carried out in any type of reactor provided that the parameters of the process are selected to allow it to be carried out under physical conditions as regards oxygen.

The examples use the apparatus described, but it is not limiting.

The following examples illustrate the invention without in any way limiting its scope. In the examples, the degree of conversion (TT) corresponds to the ratio between the number of moles of substrate transformed and the number of moles of substrate used.

The yield (RR) corresponds to the ratio between the number of moles of product formed and the number of moles of substrate used.

### EXAMPLE 1

### Preparation of p-Vanillic Acid

190 g of vanillin, 1900 g of water and 560 g of an aqueous 30% w/w sodium hydroxide solution were introduced into a 3200 ml stainless steel reactor with a diameter of 150 mm provided with a stirring system (central and counter-blade stirring).

Stirring was carried out using a stirrer with 4 inclined blades wherein the propeller was positioned so that it was one third of the depth of the liquid in the reactor off the bottom thereof.

It was stirred and 10 g of a catalyst comprising palladium deposited on carbon (activated charcoal) in an amount of 3% by weight with respect to the total catalyst weight and 0.325 g of Bi₂O₃ were introduced.

The reactor was purged with nitrogen prior to stirring.

The reactor was then heated to 95° C. at a stirring rate of 700 rpm.

A 50 g/h stream of air was then introduced via an immersed tube, maintaining a pressure of 1.5 bars in the reactor.

The reaction had to be carried out under a diffusion regime.

When it was noted that no more oxygen was being consumed, the reaction was stopped.

The reactor was purged with a stream of nitrogen.

The temperature was allowed to drop to about 50° C. and the reaction medium was filtered.

High performance liquid chromatographic analysis recorded a TT of 97.4% and an RR for p-vanillic acid of 95.4%.

The catalyst could be used again under the same conditions without adding Bi₂O₃.

High performance liquid chromatographic analysis recorded a TT of 97.6% and an RR for p-vanillic acid of 95.5%.

### EXAMPLE 2

### Preparation of Veratric Acid

190 g of veratric aldehyde, 1900 g of water and 336 g of an aqueous 30% w/w sodium hydroxide solution were introduced into a 3200 ml stainless steel reactor.

It was stirred and 10 g of 3% Pd/C catalyst and 0.325 g of Bi₂O₃ were introduced.

The reactor was purged with nitrogen prior to stirring.

The reactor was then heated to 95° C. at a stirring rate of 700 rpm.

A 50 g/h stream of air was then introduced, maintaining a pressure of 1.5 bars in the reactor.

The reaction had to be carried out under diffusion regime.

When it was noted that no more oxygen was being consumed, the reaction was stopped.

The reactor was purged with a stream of nitrogen and the temperature was allowed to drop to about 50° C.

The reaction medium was filtered.

High performance liquid chromatographic analysis recorded a TT of 97% and an RR for veratric acid of 95.2%.

The catalyst could be used again under the same conditions without adding Bi₂O₃.

No drop in activity or selectivity was observed.

### EXAMPLE 3

Example 1 was repeated, using 190 g of o-vanillic acid.

Under these conditions, the following results were obtained: TT of 99% and RR for o-vanillic acid of 95%.

### Comparative Example 4

Preparation of p-Vanillic Acid

Example 1 was repeated, using a stirring rate of 1000 rpm. In this example, a chemical regime was established.

Catalyst deactivation was observed and the results were: TT=2% and RR for vanillic acid=1.8%.

1. A process for oxidising an aromatic aldehyde to the corresponding carboxylic acid, consisting of carrying out the oxidation, in a basic medium, of an aromatic aldehyde using molecular oxygen or a gas containing molecular oxygen in the presence of a catalyst, characterized in that oxidation is carried out in the presence of an effective quantity of a catalyst based on palladium and/or platinum under conditions such that oxidation occurs in a diffusion regime.

2. A process according to claim 1, characterized in that the aromatic aldehyde has general formula (I): 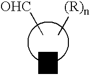
in which:
A denotes the residue of a cyclic group forming all or a portion of an aromatic, monocyclic or polycyclic carbocyclic or heterocyclic system comprising at least one formyl group;
R represents a hydrogen atom or one or more substituents which may be identical or different;
n, the number of substituents in the cyclic group, is 5 or less.

3. A process according to claim 2, characterized in that the aromatic aldehyde has formula (I) in which A represents a benzene residue or a naphthalene residue or a residue of a nitrogen-containing heterocycle, preferably pyridine, pyrimidine, pyrazine, quinoline or isoquinoline.

4. A process according to claim 2, characterized in that the aromatic aldehyde has formula (I) in which R, which may be identical or different, represents a hydrogen atom, an alkyl, alkoxy, alkenyl, alkenyloxy, hydroxyalkyl, alkoxyalkyl, cycloalkyl, cycloalkyloxy, aryl, aryloxy, arylalkyl, arylalkyloxy group, a hydroxyl group, a nitro group, a halogen atom, a halogeno group or a perhalogenoalkyl group.

5. A process according to claim 2, characterized in that n is 1 or 2.

6. A process according to claim 1, characterized in that the aromatic aldehyde has formula (Ia): 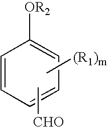
in which:
n is 4 or less, preferably 0 or 1;
R₁ represents a hydrogen atom or one or more substituents, which may be identical or different;
R₂ represents a hydrogen atom or an alkyl, alkenyl, cycloalkyl, aryl or arylalkyl group;
groups R₁ and R₂ and the 2 successive atoms on the benzene ring can together form a cyclic group containing 5 to 7 atoms, optionally comprising a further heteroatom;
two groups R₁ placed on two neighbouring carbon atoms can form a cyclic group containing 5 to 7 atoms together with the carbon atoms carrying them.

7. A process according to claim 6, characterized in that the aromatic aldehyde has formula (Ia) in which R₂ represents a hydrogen atom or a linear or branched alkyl group containing 1 to 4 carbon atoms, preferably a methyl or ethyl group or a phenyl group.

8. A process according to claim 6, characterized in that the aromatic aldehyde has formula (Ia) in which R, which may be identical or different, represents:
a hydrogen atom;
a linear or branched alkyl group containing 1 to 6 carbon atoms, preferably 1 to 4 carbon atoms such as methyl, ethyl, propyl, isopropyl, butyl, isobutyl, sec-butyl or tert-butyl;
a linear or branched alkoxy group containing 1 to 6 carbon atoms, preferably 1 to 4 carbon atoms, such as methoxy, ethoxy, propoxy, isopropoxy, butoxy, isobutoxy, sec-butoxy, tert-butoxy;
a halogen atom, preferably a fluorine, chlorine or bromine atom, or
a trifluoromethyl group.

9. A process according to claim 6, characterized in that the aromatic aldehyde has formula (Ia) in which R₂ represents a hydrogen atom or a linear or branched alkyl group containing 1 to 4 carbon atoms, preferably a methyl or ethyl group.

10. A process according to claim 6, characterized in that the aromatic aldehyde has formula (Ia) in which the formyl group is in the position meta or para to a hydroxyl group, if present on the benzene ring.

11. A process according to claim 6, characterized in that the aromatic aldehyde has formula (Ia) in which groups R₁ represent a hydrogen atom, a hydroxyl group or a linear or branched alkoxy group containing 1 to 6 carbon atoms, preferably 1 to 4 carbon atoms, and m is 0, 1 or 2 and groups OR₂ and R₁ form a methylenedioxy or ethylenedioxy group.

12. A process according to claim 1, characterized in that the aromatic aldehyde with formula (I) or (Ia) is p-methoxybenzaldehyde, vanillin, o-vanillin, isovanillin, ethylvanillin, veratric aldehyde, piperonal, protocatechuic aldehyde or 2-formyl-6-hydroxynaphthalene.

13. A process according to claim 1, characterized in that the platinum and/or palladium catalyst is supplied in the form of platinum black, palladium black, platinum oxide, palladium oxide or the noble metal itself deposited on different supports such as carbon black, graphite, activated charcoal, aluminas or activated silicas or equivalent materials, preferably carbon black.

14. A process according to claim 1, characterized in that the quantity of catalyst used, expressed as the weight of metal M₁ with respect to that of the compound with formula (I), can vary from 0.001% to 10%, preferably 0.002% to 2%.

15. A process according to claim 1, characterized in that an activator from group 1b and 8 metals is used, such as cadmium, bismuth, lead, silver, tin or germanium, preferably bismuth.

16. A process according to claim 15, characterized in that the activator is an organic or inorganic bismuth derivative selected from the group formed by: bismuth oxides; bismuth hydroxides; bismuth or bismuthyl salts of mineral hydracids such as the chloride, bromide, iodide; bismuth or bismuthyl salts of mineral oxyacids such as the sulphite, sulphate, nitrite, nitrate, phosphite, phosphate, pyrophosphate, carbonate, perchlorate; bismuth or bismuthyl salts of aliphatic or aromatic organic acids such as the acetate, propionate, salicylate, benzoate, oxalate, tartrate, lactate, or citrate; and bismuth or bismuthyl phenates, preferably the gallate or pyrogallate.

17. A process according to claim 16, characterized in that the bismuth derivative is selected from the group formed by: bismuth oxides Bi₂O₃ and Bi₂O₄; bismuth hydroxide Bi(OH)₃; bismuth chloride BiCl₃; bismuth bromide BiBr₃; bismuth-iodide BiI₃; neutral bismuth sulphate Bi₂(SO₄)₃; neutral bismuth nitrate Bi(NO₃)₃, 5H₂O; bismuthyl nitrate BiO(NO₃); bismuthyl carbonate (BiO)₂CO₃,0.5 H₂O; bismuth acetate Bi(C₂H₃O₂)₃; bismuthyl salicylate C₆H₄CO₂(BiO)OH.

18. A process according to claim 15, characterized in that the quantity of activator, expressed with respect to the weight of metal M₁ used, is between 0.1% and 100%, preferably about 50%.

19. A process according to claim 1, characterized in that the pH of the reaction is in the range 10 to 12.

20. A process according to claim 19, characterized in that the basic agent used to regulate the pH is sodium hydroxide.

21. A process according to claim 19, characterized in that the quantity of base employed is the quantity necessary to form the salt of the carboxylic function formed and to form the salt of the hydroxyl function when the compound with formula (I) or (Ia) contains one, or any other salt-forming function on the aromatic cyclic group.

22. A process according to claim 1, characterized in that the oxidation temperature is selected to be between 20° C. and 140° C., preferably between 30° C. and 100° C.

23. A process according to claim 1, characterized in that the pressure is atmosphere pressure.

24. A process according to claim 1, characterized in that the stirring conditions are such that the reaction conditions constitute diffusion regime.

25. A process according to claim 1, characterized in that it consists of introducing the aldehyde with formula (I) or (Ia), the basic agent, the palladium and/or platinum based catalyst, and optional activator.

26. A process according to claim 1, characterized in that it consists of introducing water, the basic agent, the palladium and/or platinum based catalyst, optional activator and compound with formula (I) or (Ia).

27. A process according to claim 25 or claim 26, characterized in that the reaction mixture maintained in a stream of inert gas (preferably nitrogen) is heated to the desired reaction temperature then oxygen or an oxygen-containing gas is introduced.

28. A process according to claim 27, characterized in that the medium is stirred at the desired temperature until a quantity of oxygen corresponding to that necessary to transform the formyl group into a carboxylic group has been consumed.

29. A process according to claim 1, characterized in that the aromatic acid formed is recovered after acid treatment.
